(19) United States
(12) Patent Application Publication
(10) Pub. No.: US 2024/0367181 A1
(43) Pub. Date: Nov. 7, 2024

(54) ADIPOSE TISSUE PROCESSING DEVICE

(71) Applicant: DSM IP Assets B.V., Heerlen (NL)

(72) Inventors: William T. FISHER, Exton, PA (US); Amit DEKA, Exton, PA (US)

(21) Appl. No.: 18/686,460

(22) PCT Filed: Aug. 26, 2022
(86) PCT No.: PCT/US2022/041609

§ 371 Date: Feb. 26, 2024

Related U.S. Application Data
(60) Provisional application No. 63/237,572, filed on Aug. 27, 2021.

(51) Int. Cl. B04B 1/02; B04B 1/14; B04B 3/00

(52) U.S. Cl. CPC B04B 1/02; B04B 1/14; B04B 3/00

(57) ABSTRACT

Disclosed herein are devices and methods for processing biologic tissues, such as adipose tissue or whole blood, via centrifugation, An annular cavity is present, typically at the widest interior point of a rotatable chamber. The device is operable such that a sample of biologic tissue present in the rotatable chamber can be stratified into at least two constituent layers as a function of the differing specific gravities of the constituents and at least a portion of one of the constituent layers captured in the annular cavity.

### CROSS-REFERENCE TO RELATED APPLICATIONS

This application is an International Application claiming priority to U.S. Provisional App. No. 63/237,572, filed 27 Aug. 2021, which is hereby incorporated by reference in its entirety.

### FIELD OF THE INVENTION

The present invention pertains to methods and devices for retrieving adipose-derived stem cells from adipose tissue.

### BACKGROUND

Adipose tissue, also called fat or fat tissue, is composed mostly of adipocytes. Other cell types are present, collectively termed stromal vascular fraction (SVF) cells. The SVF includes multiple cell types, including adipose-derived stem cells (ADSCs).

ADSCs are stem cells obtained from adipose tissue. ADSCs have also been described as adipose-derived stem/stromal cells, adipose-derived adult stem cells, adipose-derived adult stromal cells, adipose-derived stromal cells, adipose stromal cells, adipose mesenchymal stem cells, lipoblast, pericyte, preadipocyte, adipose stem cells, and processed lipoaspirate cells. ADSCs are known to be useful in various medical procedures to assist in the healing of an affected area of a patient, for example by providing enhanced cellular regeneration of a treatment site.

ADSCs can be obtained from adipose tissue in a multi-step process: fat tissue is extracted from the patient, the ADSCs are liberated from the fat tissue, and the ADSCs are recovered by separating them from other components of the fat tissue. Fat tissue is extracted from a patient using known techniques, such as surgery or liposuction. Existing techniques to liberate ADSCs utilize enzymes to break down the adipose tissue. The cells are then separated, usually by centrifuge, sedimentation or filtration techniques. The separated cells are usually then washed to remove the enzyme (residuals) used to treat the fat sample. Disadvantageously, the process to prepare a useful sample of ADSCs using presently available techniques takes several hours (and in some cases up to 14 days), making the ad-hoc use of such a procedure difficult or impossible. In addition, the techniques require multiple processing steps, thereby increasing the potential for contamination.

Mechanical separation and extraction of components of adipose tissues is a possible alternative to existing techniques. A centrifuge device for obtaining ADSCs is disclosed in WO2015/117007. In certain disclosed embodiments, the act of centrifugation is used to both liberate the ADSCs from a fat tissue sample and separate the ADSCs from other material once liberated.

Further examples of potentially relevant devices are disclosed in WO2012/006587, WO2012/067658, WO2013/106655, WO2013/123216, US20140021147, WO2014/011213, WO2014/0164815, WO2014/039697, WO2014/110448, WO2014/154990, US2015093362, WO2015/035221, and WO2018/044791.

### SUMMARY

The inventors have discovered that the forces that may be required to liberate and separate ADSCs from other material via centrifugation may destroy the ADSCs in the collection process. There is thus a need for improved devices and processes for liberating, separating, and collecting ADSCs from fat tissue. The disclosed devices and methods may also have utility in separation and extraction of components of other biologic tissues, such as blood.

In an embodiment, a device for processing a biologic tissue comprises:
a. a rotatable chamber arranged to rotate about an axis, the rotatable chamber at least partially defined by a first section and a second section, each of the first section and the second section comprising a base, an end, and a sidewall connecting the base and the end, the sidewall comprising at least one inclined surface such that each section decreases in interior width over at least a portion of the distance from its base to its end, the first section and second section positioned opposite one another such that the first section base and the second section base are positioned adjacent to one another; and
b. a drive unit configured to rotate the rotatable chamber about the axis and produce a centrifugal field via rotation of the rotatable chamber about the axis; wherein at a first position the device comprises a passage defining the entrance to an annular cavity, the annular cavity being present i) in a sidewall of the first section or second section, or ii) between the first section and the second section, and wherein the device is configured such that upon operation at least a portion of a sample of biologic tissue present in the rotatable chamber is stratified into at least two constituent layers as a function of the differing specific gravities of the constituents of the sample of biologic tissue, and wherein at least a portion of one or more of the at least two constituent layers may enter the annular cavity.

The disclosed inventions may be beneficial over the prior art in that they may extract a greater number of cells from a sample or at a greater percentage of viability, be more sterile, more self-contained, faster, more compact, easier to use, better at removing contaminants, better at removing oils or other liquids, or otherwise more efficacious than the prior art devices.

### BRIEF DESCRIPTION OF THE DRAWINGS

FIG. 1 is a perspective view of a cross-section of an embodiment of the invention.

FIG. 2 is a perspective detail view of a portion of the embodiment of FIG. 1.

FIG. 3 is a cross-section view of an embodiment of the invention.

FIG. 4 is a cross-section view of an embodiment of the invention comprising multiple screens and associated rollers.

FIG. 5 is a perspective view of an insert for use with an embodiment of the invention.

### DESCRIPTION

The device comprises a rotatable chamber. The rotatable chamber is arranged to rotate around a central axis. In an embodiment, the axis is the longitudinal axis of the rotatable chamber. In an embodiment, the rotatable chamber generally may be divided in two sections. A first section is generally present farther from the drive unit while a second section is present closer to the drive unit. In an embodiment, the first section and second section are separated by the annular cavity, such that the first section is the part of the rotatable chamber above the annular cavity and the second section is the part of the rotatable chamber that is between the drive unit and the annular cavity.

Although the first section and second section may be referred to herein as separate parts of the rotatable chamber, this does not mean that the rotatable chamber must be able to be physically broken down into separate first and second sections. In an embodiment, the rotatable chamber may be physically separable into a first section and a second section. In an embodiment, the rotatable chamber may be present as a single piece.

The rotatable chamber comprises a sidewall. In an embodiment, each of the first section and the second section comprise a sidewall. The sidewall connects the ends of the rotatable chamber, or the base and the end of each section. The sidewall is generally dimensioned to direct the sample into the annular cavity upon centrifugation. Accordingly, the sidewall comprises an interior surface over at least a portion of its length such that the width of the rotatable chamber is not constant over at least a portion of the distance of the inclined surface. Additional surfaces may be present, for example, an inner surface that is parallel to the axis extending a portion of the distance from the first end to the second end. The sidewall and/or rotatable chamber may be fully or partially transparent to allow for visual inspection of the sample and centrifugation.

The inclined surface urges the constituents of the sample to move toward the annular cavity. In an embodiment, the rotatable chamber has a lesser width at each end and a greater width at or near its midsection. In an embodiment, each of the first section and the second section comprises the shape of the frustum of a cone. In an embodiment, the rotatable chamber is disc-like. In an embodiment, the largest radial distance from the axis to the sidewall in the interior of the rotatable chamber is in the annular cavity. In an embodiment, the largest distance from the axis in the rotatable chamber is between the first end and the second end. In an embodiment, the largest radial distance from the axis to the sidewall in the interior of the rotatable chamber is proximate where the first section and the second section are nearest one another. In an embodiment, the largest distance from the axis in the rotatable chamber is between 25% and 75%, 40% to 60%, 45% to 55%, or 50% of the distance from the first end to the second end along the axis.

In an embodiment, the inclined surface may be straight or curved. In an embodiment, the sidewall is uniformly angled from the end of a section to its base. In an embodiment, the sidewall is angled at multiple different angles along its length from the base to the end of a section. In an embodiment, at least a portion of the sidewall is curved.

In an embodiment, the device comprises an outer enclosure surrounding the rotatable chamber. The rotatable chamber may be contained within an outer enclosure to ensure that the operator cannot inadvertently touch the rotatable chamber during operation. The outer enclosure may be arranged to receive a biologic mixture from the rotatable chamber and may be arranged coaxially upon the central axis of the rotatable chamber. In an embodiment, the outer enclosure encloses at least the rotatable chamber and the collection container.

The rotatable chamber may comprise an inlet to allow a sample, e.g. lipoaspirate, to be placed into the rotatable chamber. In an embodiment, the inlet is configured to receive the tip of a syringe or catheter. In an embodiment, the inlet comprises a cap that can be opened to allow input of the sample and closed to seal the rotatable chamber or outer enclosure. In an embodiment, the rotatable chamber incorporates one or more vents to allow for fluid displacement. One or more vents may provide for air to enter the chamber from which a fluid is displaced, or for air to leave a chamber from which fluid enters.

The rotatable chamber comprises an annular cavity. The annular cavity is a ring-like cavity that is in fluid communication with the interior of the rotatable chamber. In an embodiment, the purpose of the annular cavity is to capture the densest portion of the sample stratified within the rotatable chamber. Some or all of such densest portion may fill the annular cavity upon operation of the device. Such densest portion may remain in the annular cavity after stopping the rotation of the rotatable chamber or may be extracted during rotation. A passage from the interior of the rotatable chamber to the annular cavity may be present. In an embodiment, the annular cavity is present proximate to and wider than the widest point of the interior of the rotatable chamber when the passage is closed. In an embodiment, the passage begins at the greatest width of the interior volume of the rotatable chamber.

Use of an annular cavity as described may have the principal advantage that ADSCs are not subject to forces that may affect the viability of the ADSCs, such as by ejecting them into a collection container. In this regard, the annular cavity may operate as an improvement in that the densest portion of the sample may gently migrate up and/or down the sidewall of the rotatable chamber and into the annular cavity during rotation rather than contacting a surface at high velocity.

Typically, the annular cavity comprises the shape of a circular or ellipsoidal ring. The annular cavity may extend entirely around the perimeter of the rotatable chamber or may extend over only part of the perimeter of the rotatable chamber. In an embodiment, the annular cavity has an obstruction within it in order to fully or partially disrupt fluid flow around the annular cavity. In an embodiment, the annular cavity is centered about the axis or may be off-center from the axis. In an embodiment, the annular cavity has a uniform depth. In an embodiment, the annular cavity has a depth that varies along its length. In an embodiment, the annular cavity extends outward, approximately perpendicular from the axis, along its circumference. In an embodiment, the annular cavity extends in a direction away from the axis and toward either the first end or second end.

The annular cavity may be formed in numerous ways. In an embodiment, the annular cavity forms part of a sidewall of the rotatable chamber, such as a sidewall of the first section or second section. In an embodiment the annular cavity is present in the sidewall of the first or second section proximate the base of said section, i.e. proximate to where the first section and the second section meet. In an embodiment, the annular cavity is partially formed in each of the first and second section such that the annular cavity is fully formed when the first and second section are connected.

In an embodiment, the sections of the rotatable chamber are connected via a collar and the collar comprises the annular cavity. Thus, in an embodiment, the device comprises a collar at the widest point of the rotatable chamber, the collar comprising the annular cavity. In an embodiment, the collar forms part of the first section and/or the second section.

Fluid communication between the interior of the rotatable chamber and the annular cavity may be established by, for example, the actuation of a first valve. Such first valve may be openable by manual or electronic action. In an embodiment, the first valve is openable in response to the centrifugal force exerted on it by rotation of the chamber. In an embodiment, the first valve is manually operable by the user. In an embodiment, the first valve is normally in the closed position, thereby restricting flow to the annular cavity, and opens in response to the force exerted on a dynamic seal, for instance, a rubber o-ring or biasing spring. In an embodiment, the device is operable to move from the first position to the second position by closing a first valve operable to control fluid communication through the passage to the annular cavity.

In an embodiment, the valve may be formed by the collar, for example, by opening the passage to the annular cavity due to the movement of the first section or second section as allowed by the collar. In an embodiment, the collar constrains motion of the first section and second section in directions normal to the axis and allows motion of the first or second section in the direction of the axis. In an embodiment, the device is movable from the second position to the first position upon translating the first section and/or second section axially along the axis. Thus, in a first position the annular cavity may be in fluid communication with the interior of the rotatable chamber and in a second position the annular cavity may not be in fluid communication with the interior of the rotatable chamber.

Movement of the first section or second section may be controlled by, for example, a biasing element such as an o-ring or spring, that may be present that allows axial movement of the first section or the second section due to the centrifugal force achieved by sufficient rotation of the rotatable chamber. In an embodiment, the passage is then closed automatically as the speed of the device is reduced to a certain level. In an embodiment, the device may move from the first position to the second position in response to user input.

In an embodiment, the annular cavity comprises a port for providing access to the material present in the annular cavity from the exterior of the rotatable chamber. In an embodiment, the port is openable to discharge material from the annular cavity into a collection container. In an embodiment, the port is in fluid communication with a collection container. In an embodiment, the greatest depth of the annular cavity is proximate a port in the annular cavity.

In an embodiment, the annular cavity comprises two ports, one on either side of an obstruction that inhibits fluid flow around the annular cavity. Such configuration allows for a container, e.g. a syringe to be present at each port. For example, one syringe may contain a sterile fluid, e.g. saline, while the other is empty. Flushing the sterile fluid into the annular cavity may force the collected portion of the sample into the empty syringe, thereby obtaining the sample. The collection containers may be filled while the device is running or after the device is stopped.

The rotatable chamber may comprise a first outlet. The first outlet is typically located closer to the axis than the annular cavity. In an embodiment, the first outlet extends through the sidewall from the interior of the rotatable chamber to the exterior of the rotatable chamber.

In an embodiment, the first outlet is in fluid communication with a first outlet valve that determines whether liquid may flow into the tube from the first outlet. In an embodiment, the valve is manually operable by the user. In an embodiment, the valve operates automatically as a result of the operation of the device. In an embodiment, the valve comprises a dynamic seal. A dynamic seal is a seal that is normally in the closed position, thereby restricting flow through the first outlet, and opens in response to the force exerted on the dynamic seal. The force may be exerted on the dynamic seal by the sample or may be exerted on the dynamic seal by the rotation of the rotatable chamber. In an embodiment, the dynamic seal comprises an o-ring positioned in the first outlet.

In an embodiment, a collection container collects material that is expelled by centrifugation of the sample. In an embodiment, the device comprises a collection container. In an embodiment, a collection container is contained within the device or within the outer enclosure. In an embodiment, the collection container is separately removable from the device. In an embodiment, the collection container comprises an annular shape such that it fits around the rotatable chamber. In an embodiment, the collection container is or may become in fluid communication with the annular channel. In an embodiment, the collection container is or may become in fluid communication with the first outlet.

In an embodiment, the rotatable chamber further comprises a second outlet at a different radial distance from the axis than the first outlet. The second outlet may comprise a second valve. Further outlets and valves may also be present.

The rotatable chamber may further comprise one or more screens, such as a morselizing screen or a retaining screen, in its interior. A morselizing screen is a screen configured to break apart a sample, such as to liberate a certain type of cells or reduce the size of the sample into smaller portions. Generally, a morselizing screen allows for portions of the sample to pass through the morselizing screen, perhaps after urging the portions through the morselizing screen such as with a roller, whereas a retaining screen allows for portions of the sample to be retained on the surface of the retaining screen nearer the axis.

In an embodiment, the rotatable chamber further comprises a morselizing screen. The morselizing screen is configured to morselize tissue into smaller fragments. In an embodiment, the act of morselizing comprises breaking a plurality of adipocytes present in adipose tissue. A morselizing screen may be useful for, for example, extracting multipotent stem cells, such as ADSCs, from adipose tissue.

In an embodiment the morselizing screen is cylindrical. In an embodiment, a surface of the morselizing screen is substantially parallel to an inner surface of the sidewall. In an embodiment, the morselizing screen is frustoconical. In an embodiment, the morselizing screen rotates along with the rotatable chamber. In an embodiment, the morselizing screen is stationary relative to the rotatable chamber. In an embodiment, the morselizing screen rotates at a revolution frequency that is less that the revolution frequency of the rotatable chamber. In an embodiment, the morselizing screen projects away from an end. In an embodiment, the morselizing screen extends concentrically around the axis of the rotatable chamber. In an embodiment, the retaining screen is mesh-like. The morselizing screen may be a metal or polymer wire material, or a perforated sheet having openings of sufficient size to at least partially morselize the sample.

Although the purpose of the morselizing screen is to morselize the tissue, the tissue may not immediately morselize when it comes into contact with the morselizing screen. The tissue may instead be somewhat retained by the morselizing screen. It may be advantageous to urge the tissue through the morselizing screen in order to morselize the tissue. In an embodiment, the tissue is urged away from the axis of rotation of the rotatable chamber and toward the sidewall. In an embodiment, the device further comprises a roller arranged to urge tissue through the morselizing screen. In an embodiment, the roller comprises a roller axle and a cylinder rotatable about the roller axle. In an embodiment, the roller axle is formed of stiff wire that extends through the center bore of the cylinder and the wire is mounted so that it may be secured in a stationary position within the rotatable chamber. In an embodiment, the roller axle comprises molded plastic.

In an embodiment, the roller is present proximate or in contact with the morselizing screen. As either the roller axle or morselizing screen rotate about the axis of rotation of the rotatable chamber along with the rotatable chamber, the roller may come into contact with tissue built up on the interior surface of the morselizing screen, thereby urging the tissue through the morselizing screen. In an embodiment, the morselizing screen is rotatable while the roller axle remains stationary. In an embodiment, the roller is present near the inner surface of the morselizing screen but does not contact the morselizing screen. In an embodiment, the roller rolls against the inner surface of the morselizing screen.

In an embodiment, the device comprises a second or further morselizing screen. The second or further morselizing screen is intended to further morselize the sample after it passes through a previous morselizing screen or screens. As such, the second morselizing screen is present farther from the axis than the first morselizing screen. In an embodiment, the second morselizing screen comprises an average aperture size that is less than the morselizing screen. The second morselizing screen may be associated with a second roller. In an embodiment, the device further comprises a third morselizing screen, the third morselizing screen having an average aperture size that is less than the second morselizing screen, the third morselizing screen present at a greater distance from the axis than the second morselizing screen. In an embodiment, the one or more morselizing screens are attached to the second section and one or more roller axles are attached to the first section. In an embodiment, the roller axles are present on one or more separate pieces that do not rotate along with the rotatable chamber. In order for the device to be properly balanced during rotation, in an embodiment each roller is evenly spaced circumferentially around the axis. For example, three rollers would each be present 120 degrees apart about the axis.

In an embodiment, the rotatable chamber further comprises a retaining screen. The retaining screen is configured to substantially retain a certain type of tissue on the interior of the retaining screen while allowing liquids to pass through. A retaining screen may serve to keep obstructions in the form of biologic tissue from reaching the annular cavity or outlet(s). In an embodiment, the retaining screen is configured to retain fat tissue on its inner surface. In an embodiment the retaining screen is cylindrical. In an embodiment, a surface of the retaining screen is substantially parallel to an inner surface of the sidewall. In an embodiment, the retaining screen is frustoconical. In an embodiment, the retaining screen rotates along with the rotatable chamber. In an embodiment, the retaining screen is stationary relative to the rotatable chamber. In an embodiment, the retaining screen projects away from the base. In an embodiment, the retaining screen extends concentrically around the axis of the rotatable chamber. In an embodiment, the retaining screen is mesh-like. The retaining screen may be a metal or polymer wire material, or a perforated sheet having openings of sufficient size to allow for the passage of fluid while inhibiting the passage of other constituents of the sample.

In an embodiment, the rotatable chamber comprises a trap. A trap is region at the bottom of the rotatable chamber that traps certain constituents of the sample during or after rotation of the rotatable chamber such that the trapped constituents are inhibited from remixing with other constituents of the sample after rotation is stopped. In an embodiment, the rotatable chamber comprises an oil trap for trapping oil near the base of the rotatable chamber.

In an embodiment, a filter is positioned at a distance above an end of the rotatable chamber, thereby forming a trap. The filter may be useful to keep the constituent of biologic tissue separate from any oil or lipid fraction that may have been separated by the centrifugation. For example, when the rotation is stopped the second portion of oil and lipid fraction may undesirably remix with the adipose tissue if the filter is not present. The filter serves to keep the second portion of the oil and lipid fraction separate from the adipose tissue because the oil and lipid fraction will pass through the filter due to gravity whereas the adipose tissue may not pass through the filter.

In an embodiment, the filter is shaped like a disc. In an embodiment, the filter is made of metal, such as stainless steel. In an embodiment, the filter is made of a plastic, such as nylon. In an embodiment, the filter has an average opening size is of at least 0.025 mm or 0.05 mm. In an embodiment, the filter to has an average opening size of at most 0.25 mm, 0.2 mm, 0.15 mm, or 0.13 mm.

The rotatable chamber may comprise a fiber collector as disclosed in WO2020/077097. The fiber collector is configured to collect fibers from the sample, such as collagen fibers in lipoaspirate. A wiper for disrupting fibers hanging off of the fiber collector may also be present.

In an embodiment, the device comprises a drive unit, which serves as a rotation source for the rotatable chamber. Upon reaching a sufficient rotation speed, a biologic tissue sample stratifies into at least two constituents based on the density of the constituents. The drive unit preferably couples to the rotatable chamber and/or outer enclosure. The drive unit comprises means to rotate the rotatable chamber. The drive unit preferably comprises an electric motor configured to rotate the rotatable chamber, but may also contains a hand crank or any other means to rotate the rotatable chamber that may be known to a person skilled in the art. In an embodiment, the drive unit comprises a hand crank and a spring and is configured so that the drive unit can be would up and the spring released to rotate the rotatable chamber. In an embodiment, the drive unit is separable from the rotatable chamber, such that the drive unit may be reused without substantial cleaning or sterilization. In an embodiment, the rotatable chamber, and optionally the outer enclosure, are single-use or may be cleaned and sterilized, such that they may be reused.

In an embodiment, the device comprises two units: a processing unit and a drive unit. The drive unit comprises the base of the unit and rotation source. The processing unit comprises the upper portion of the device and comprises the rotatable chamber. The processing unit is generally single use, though it may be sterilized and reused, and the drive unit is generally reusable without sterilization.

In an embodiment, the drive unit is configured to rotate the rotatable chamber at multiple speeds. In an embodiment, the drive unit is configured to rotate the rotatable chamber such that the g-force acting on a sample within the rotatable chamber is 1000 g or less, 8000 g or less, or 5000 g or less. In an embodiment, the drive unit is configured to oscillate the rotatable chamber, such as a washing machine. In an embodiment, the drive unit is configured to oscillate in a sinusoidal wave pattern. In an embodiment, the drive unit is configured to oscillate in a square wave pattern. The drive unit may comprise manually operated controls, such as buttons, to allow an operator to direct the drive unit to rotate the rotatable chamber in a certain way, such as at a certain direction or certain speed.

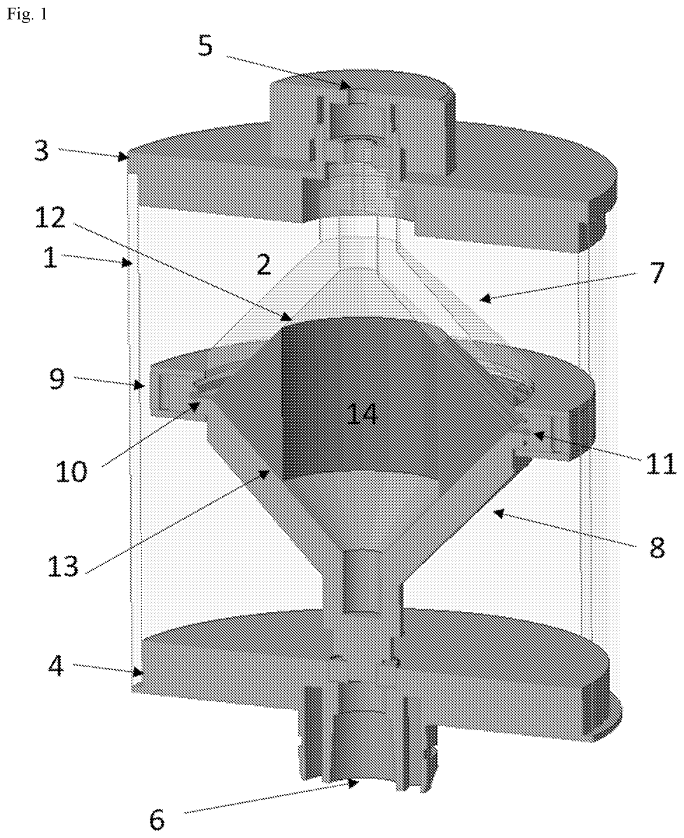

FIG. 1 depicts a perspective cross-section view of an embodiment of a tissue processing device in operation. The device comprises an outer chamber 1. The outer chamber encloses rotatable chamber 2. The outer chamber is transparent and is bounded by a first outer chamber end 3 and a second outer chamber end 4. The first end 3 of the outer chamber comprises an inlet 5 to access the interior of rotatable chamber 2. The second end 4 comprises an interface to a drive unit 6, the drive unit (not pictured) being operable to rotate the rotatable chamber 2.

Rotatable chamber 2 comprises a first section 7 and a second section 8. Each section comprises a base and an end. First section 7 end is proximate the first outer chamber end 3. Second section 8 end is proximate second outer chamber end 4. As depicted, the base of each section meets approximately at the midline of the rotatable chamber and are connected by collar 9. The base of each section and the collar together form a passage 10 leading to annular cavity 11.

The rotatable chamber comprises sidewalls 12, 13 that are inclined such that each of the first section and second section decrease in interior width from their base to their end. During operation of the device, biologic tissue sample 14 is stratified based on the specific gravity of its constituents. The densest constituent of the biologic tissue sample 14 has filled annular cavity 11.

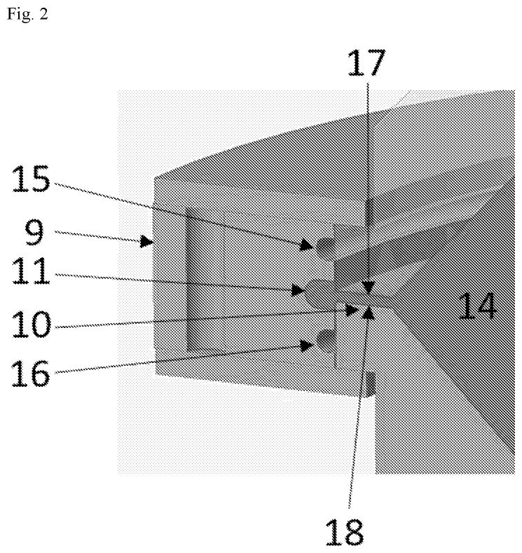

A more detailed view of collar 9 is depicted in FIG. 2. The collar may comprise o-rings 15, 16. One mode of operation involves a first position, wherein passage 10 is open (as depicted), allowing a portion of the biological tissue sample to flow into annular cavity 11. Depending on the desired mode of operation, passage 10 may be fixed in the first position or may be movable between a first position and a second position. In a second position, the passage may be closed by the mating of surface 17 of the first section base and surface 18 of the second section base. The device may reach the second position from the first position via the axial translation of either or both of the first and second section of the rotatable chamber.

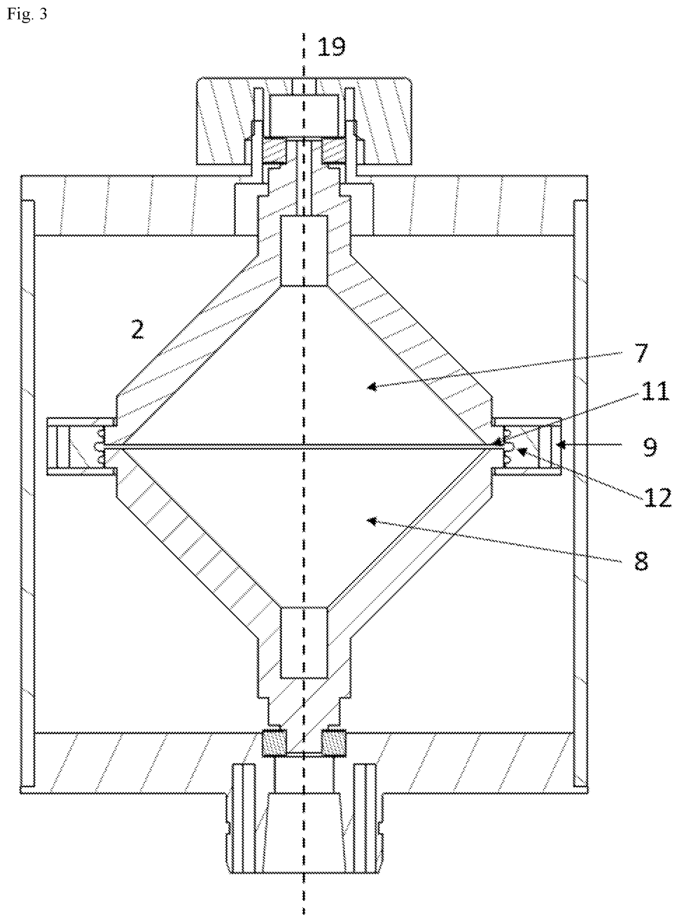

A cross-sectional view of an embodiment of the invention is shown in FIG. 3. The device is arranged to rotate about central longitudinal axis 19. First section 7 and second section 8 are arranged adjacent to and opposite one another and connected by collar 9. Passage 10 and annular cavity 11 extend fully around the inner circumference of rotatable chamber 2 between first section 7 and second section 8.

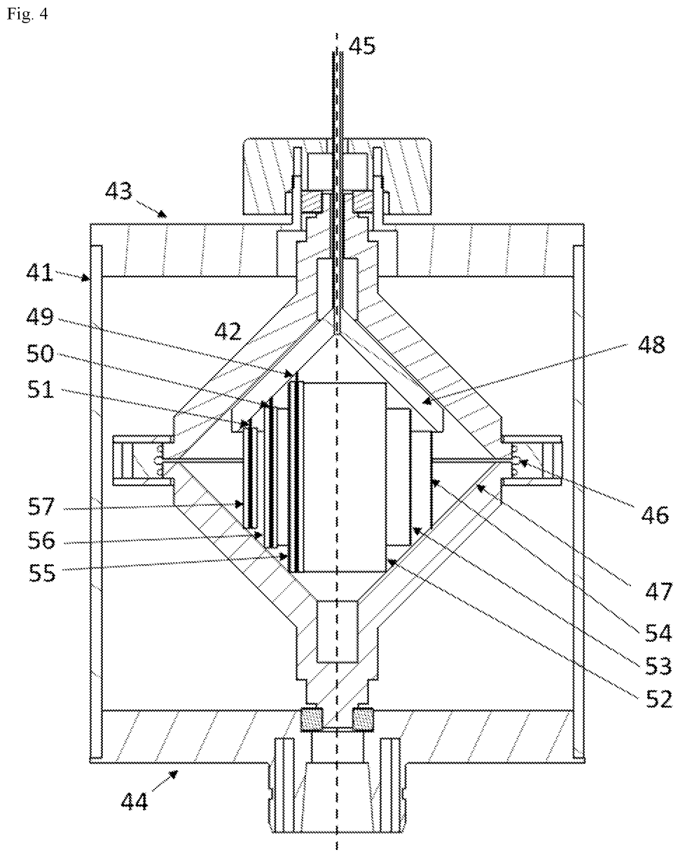

FIG. 4 is a cross-sectional view of a further embodiment of the invention. Similar to the embodiment depicted in FIG. 3, the device comprises outer chamber 41 and rotatable chamber 42. Outer chamber 41 is bounded by first end 43 and second end 44. Rotatable chamber 42 is arranged to rotate around axis 45. The device in FIG. 4 differs from the device of FIG. 3 in that the FIG. 4 device comprises screens and associated rollers to urge portions of biologic tissue through each screen. Three screens are present, first screen 52, second screen 53, and third screen 54. First screen 52 is at a lesser radial distance from axis 45 and has a greater average opening size than second screen 53. Second screen 53 is at a lesser radial distance from axis 45 and has a greater average opening size than third screen 54. The size of portions of the biologic tissue thus decreases as the biologic tissue moves from axis 45, where it is placed within rotatable chamber 42, to annular cavity 46. Each screen 52, 53, 54 is cylindrical and is in contact with sidewall 47. Biologic tissue must therefore pass through all three screens to reach annular cavity 46. Since each screen is in contact with rotatable chamber 42, the screens rotate around axis 45 along with the rotatable chamber.

Each screen 52, 53, 54 is associated with a roller, 55, 56, 57, respectively. Each roller is in contact with or held proximal to the inner surface of its associated screen in order to urge biologic tissue through the screen. Due to the sloping surface of sidewall 47, first roller 55 is longer than second roller 56, which is longer than third roller 57. Each roller is supported by insert 48, which comprises roller axles 49, 50, and 51. Roller axles 49, 50, and 51 are associated with rollers 55, 56, and 57, respectively. The roller axles 49, 50, 51 are attached to insert 48. Insert 48 is held stationary by securing it to first end 43 or another non-rotating part of the device. The rollers roll against or near each screen as the screens rotate about the axis. The rollers revolve around their roller axles due to force exerted on the outer surface of the rollers by the inner surface of its associated screen or the force exerted by biologic tissue that has built up on the screen.

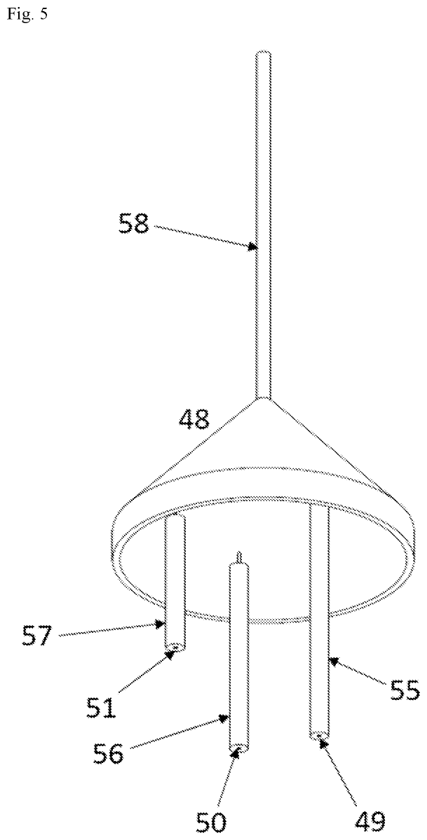

FIG. 5 is a perspective view of inert 48. Insert 48 comprises shaft 58. Shaft 58 may have an inner channel to allow for biologic tissue to be deposited into the interior of the rotatable chamber via the inner channel of shaft 58. Shaft 58 may be secured to a non-rotatable portion of the device such that the insert does not rotate along with the rotatable chamber. Roller axles 49, 50, and 51 protrude or are connected to the base of insert 48. Roller axles 49, 50, 51 are associated with cylindrical rollers 55, 56, 57, respectively.

### Additional Description of Certain Exemplary Embodiments

1. A device for processing biologic tissue comprising a rotatable chamber arranged to rotate about an axis, the rotatable chamber at least partially defined by a first section and a second section, each of the first section and the second section comprising a base, an end, and a sidewall connecting the base and the end, the sidewall comprising at least one inclined surface such that each section decreases in interior width over at least a portion of the distance from its base to its end, the first section and second section positioned opposite one another such that the first section base and the second section base are positioned adjacent to one another, and an annular cavity i) in a sidewall of the first section or second section, or ii) between the first section and the second section.
2. A device for processing biologic tissue comprising a rotatable chamber arranged to rotate about an axis and comprising a sidewall connecting the ends of the chamber, wherein the interior of the rotatable chamber varies in width along at least a portion of the distance from a first end to the second end such that the widest point of the rotatable chamber is located between the first end and the second end, the rotatable chamber comprising an annular cavity proximate its widest point.
3. A device for processing biologic tissue, the device comprising:
   a. a rotatable chamber arranged to rotate about an axis, the rotatable chamber at least partially defined by a first section and a second section, each of the first section and the second section comprising a base, an end, and a sidewall connecting the base and the end, the sidewall comprising at least one inclined surface such that each section decreases in interior width over at least a portion of the distance from its base to its end, the first section and second section positioned opposite one another such that the first section base and the second section base are positioned adjacent to one another; and
   b. a drive unit configured to rotate the rotatable chamber about the axis and produce a centrifugal field via rotation of the rotatable chamber about the axis; wherein at a first position the device comprises a passage defining the entrance to an annular cavity, the annular cavity being present i) in a sidewall of the first section or second section, or ii) between the first section and the second section, and wherein the device is configured such that upon operation at least a portion of a sample of biologic tissue present in the rotatable chamber is stratified into at least two constituent layers as a function of the differing specific gravities of the constituents of the sample of biologic tissue, and wherein at least a portion of one or more of the at least two constituent layers may enter the annular cavity.
4. A device for processing biologic tissue, the device comprising:
   a. a rotatable chamber arranged to rotate about an axis and comprising a sidewall connecting the ends of the chamber, wherein the interior of the rotatable chamber varies in width along at least a portion of the distance from a first end to the second end such that the widest point of the rotatable chamber is located between the first end and the second end, the rotatable chamber comprising an annular cavity proximate its widest point; and
   b. a drive unit configured to rotate the rotatable chamber about the axis and produce a centrifugal field via rotation of the rotatable chamber about the axis;
wherein the device is configured such that upon operation at least a portion of a sample of biologic tissue present in the rotatable chamber is stratified into at least two constituent layers as a function of the differing specific gravities of the constituents of the sample of biologic tissue, and wherein at least a portion of one or more of the at least two constituent layers may enter the annular cavity.
5. The device according to any one of the preceding exemplary embodiments, wherein at a first position the device allows fluid communication from the interior of the rotatable chamber to the annular cavity and at a second position there is no fluid communication from the interior of the rotatable chamber to the annular cavity.
6. The device according to any one of the preceding exemplary embodiments, wherein the biologic tissue is blood.
7. The device according to any one of the preceding exemplary embodiments, wherein the biologic tissue is adipose tissue.
8. The device according to any one of the preceding exemplary embodiments, wherein the device is operable to separate and retain adipose-derived stem cells from adipose tissue.
9. The device according to any one of the preceding exemplary embodiments, wherein the device is operable to liberate, separate, and retain adipose-derived stem cells from adipose tissue.
10. The device according to any one of the preceding exemplary embodiments, wherein “in fluid communication” means that fluid makes the connection between the parts specified, that the parts are connected by fluid, or that fluid may flow from one part specified to another part specified.
11. The device according to any one of the preceding exemplary embodiments, wherein the narrowest portion of the rotatable chamber is at one or both of the first end and the second end.
12. The device according to any one of the preceding exemplary embodiments, wherein the second end is the bottom of the rotatable chamber.
13. The device according to any one of the preceding exemplary embodiments, wherein the second section is positioned closer to the drive unit than the first section.
14. The device according to any one of the preceding exemplary embodiments, wherein the first end has a width that is smaller than the width of the second end.
15. The device according to any one of the preceding exemplary embodiments, wherein the axis is the central longitudinal axis of the rotatable chamber.
16. The device according to any one of the preceding exemplary embodiments, wherein the sidewall is angled in a first direction and a second direction, wherein the first direction is away from the first end, towards the second end, and away from the axis, and wherein the second direction is away from the first end, towards the second end, and towards the axis.
17. The device according to any one of the preceding exemplary embodiments, wherein the largest diameter of the rotatable chamber is present at a location on the sidewall between the first end and the second end.
18. The device according to any one of the preceding exemplary embodiments, wherein the sidewall of the first section and the sidewall of the second section define the passage therebetween.
19. The device according to any one of the preceding exemplary embodiments, wherein the first section and the second section comprise the shape of a frustum of a cone.
20. The device according to any one of the preceding exemplary embodiments, wherein the largest distance from the axis in the rotatable chamber is between the first end and the second end.
21. The device according to any one of the preceding exemplary embodiments, wherein the largest distance from the axis in the rotatable chamber is between 25% and 75%, 40% to 60%, 45% to 55%, or 50% of the distance from the first end to the second end along the axis.
22. The device according to any one of the preceding exemplary embodiments, wherein the annular cavity is a ring-like cavity in fluid communication with the interior of the rotatable chamber.
23. The device according to any one of the preceding exemplary embodiments, wherein the annular cavity is a ring-like cavity that is in fluid communication with the interior of the rotatable chamber upon actuation of a first valve.
24. The device according to any one of the preceding exemplary embodiments, wherein the first valve is openable by manual or electronic action.
25. The device according to any one of the preceding exemplary embodiments, wherein the first valve is openable in response to the centrifugal force exerted on it by rotation of the chamber.
26. The device according to any one of the preceding exemplary embodiments, wherein the first valve is manually operable by the user.
27. The device according to any one of the preceding exemplary embodiments, wherein the first valve operates automatically as a result of the operation of the device.
28. The device according to any one of the preceding exemplary embodiments, wherein the first valve comprises a dynamic seal.
29. The device according to any one of the preceding exemplary embodiments, wherein the dynamic seal is normally in the closed position, thereby restricting flow to the annular cavity, and the dynamic seal opens in response to the force exerted on the dynamic seal.
30. The device according to any one of the preceding exemplary embodiments, wherein the device is operable to move from the first position to the second position by closing a first valve operable to control fluid communication through the passage to the annular cavity.
31. The device according to any one of the preceding exemplary embodiments, wherein the annular cavity comprises the shape of a circular or ellipsoidal ring.
32. The device according to any one of the preceding exemplary embodiments, wherein the annular cavity extends fully around the circumference of the rotatable chamber.
33. The device according to any one of the preceding exemplary embodiments, wherein the annular cavity does not extend fully around the circumference of the rotatable chamber.
34. The device according to any one of the preceding exemplary embodiments, wherein the annular cavity comprises an obstruction to disrupt or prohibit fluid flow around the annular cavity.
35. The device according to any one of the preceding exemplary embodiments, wherein the annular cavity is circular or ellipsoidal, wherein the center of the annular cavity is a point on the axis.
36. The device according to any one of the preceding exemplary embodiments, wherein the annular cavity is circular or ellipsoidal, wherein the center of the annular cavity is not a point on the axis.
37. The device according to any one of the preceding exemplary embodiments, wherein the depth of the annular cavity is uniform along its circumference.
38. The device according to any one of the preceding exemplary embodiments, wherein the depth of the annular cavity varies along its circumference.
39. The device according to any one of the preceding exemplary embodiments, wherein the annular cavity has a maximum distance from the axis at a first radial location that is greater than the maximum distance from the axis at a second radial location.
40. The device according to any one of the preceding exemplary embodiments, wherein the annular cavity extends approximately perpendicular to the axis.
41. The device according to any one of the preceding exemplary embodiments, wherein the annular cavity extends in a direction away from the axis and toward either the first end or second end.
42. The device according to any one of the preceding exemplary embodiments, wherein the annular cavity comprises a port, wherein the port provides access to the material present in the annular cavity from exterior of the rotatable chamber or is openable to discharge material from the annular cavity into a collection container.
43. The device according to any one of the preceding exemplary embodiments, wherein the annular cavity comprises a port, wherein the port is in fluid communication with a collection container.
44. The device according to any one of the preceding exemplary embodiments, wherein the greatest depth of the annular cavity is proximate a port in the annular cavity.
45. The device according to any one of the preceding exemplary embodiments, wherein the annular cavity comprises an obstruction that inhibits fluid flow around the annular cavity and wherein the annular cavity has a first port proximate a first side of the obstruction and a second port proximate a second side of the obstruction.
46. The device according to any one of the preceding exemplary embodiments, wherein the annular cavity is present in the sidewall of the first section.
47. The device according to any one of the preceding exemplary embodiments, wherein the annular cavity is present in the sidewall of the second section.
48. The device according to any one of the preceding exemplary embodiments, wherein the annular cavity is present in the sidewall of the first section proximate the first section base.
49. The device according to any one of the preceding exemplary embodiments, wherein the annular cavity is present in the sidewall of the second section proximate the second section base.
50. The device according to any one of the preceding exemplary embodiments, wherein the annular cavity is present between the first section and the second section.
51. The device according to any one of the preceding exemplary embodiments, wherein the passage begins at the greatest width of the interior volume of the rotatable chamber.
52. The device according to any one of the preceding exemplary embodiments, further comprising a collar at the widest point of the rotatable chamber, the collar comprising the annular cavity.
53. The device according to any one of the preceding exemplary embodiments, further comprising a collar connecting the first section and the second section.
54. The device according to any one of the preceding exemplary embodiments, further comprising a collar connecting the first section and the second section, wherein the collar forms part of the first section and/or the second section.
55. The device according to any one of the preceding exemplary embodiments, further comprising a collar connecting the first section and the second section, wherein the collar constrains movement of the first section and second section in directions normal to the axis and allows motion of the first or second section in the direction of the axis.
56. The device according to any one of the preceding exemplary embodiments, wherein at a second position the annular cavity is not in fluid communication with the interior of the rotatable chamber and at a first position the annular cavity is in fluid communication with the interior of the rotatable chamber.
57. The device according to any one of the preceding exemplary embodiments, wherein the device is movable from the second position to the first position upon sufficient speed of rotation of the rotatable chamber about the axis.
58. The device according to any one of the preceding exemplary embodiments, wherein the device is movable from the second position to the first position upon the creation of a sufficient centrifugal field due to the rotation of the rotatable chamber.
59. The device according to any one of the preceding exemplary embodiments, wherein the device is movable from the first position to the second position upon user input.
60. The device according to any one of the preceding exemplary embodiments, wherein the device is movable from the second position to the first position upon translating the first section and/or second section axially along the axis.
61. The device according to any one of the preceding exemplary embodiments, wherein the device is configured such that after a sample of biologic tissue present in the rotatable chamber is stratified, at least a portion of constituent having the highest specific gravity is at least partially captured in the annular cavity.
62. The device according to any one of the preceding exemplary embodiments, wherein the rotatable chamber is at least partially transparent.
63. The device according to any one of the preceding exemplary embodiments, further comprising an outer enclosure enclosing the rotatable chamber.
64. The device according to any one of the preceding exemplary embodiments, wherein the outer enclosure is arranged to receive a biologic mixture from the rotatable chamber.
65. The device according to any one of the preceding exemplary embodiments, wherein the outer enclosure is arranged coaxially upon the axis of rotation of the rotatable chamber.
66. The device according to any one of the preceding exemplary embodiments, wherein the rotatable chamber comprises an inlet to allow a sample to be placed into the rotatable chamber.
67. The device according to any one of the preceding exemplary embodiments, further comprising a morselizing screen.
68. The device according to any one of the preceding exemplary embodiments, further comprising a morselizing screen configured to morselize a biologic tissue into smaller fragments, wherein the morselizing screen meets the sidewall at a second radial distance from the axis, and wherein the second radial distance is less than the radial distance from the axis to the annular cavity.
69. The device according to any one of the preceding exemplary embodiments, wherein the morselizing screen is operable to morselize tissue into smaller fragments.
70. The device according to any one of the preceding exemplary embodiments, wherein the morselizing screen is configured to break a plurality of adipocytes present in adipose tissue.
71. The device according to any one of the preceding exemplary embodiments, wherein the axis is coaxial with the central axis of the morselizing screen.
72. The device according to any one of the preceding exemplary embodiments, wherein the morselizing screen is cylindrical.
73. The device according to any one of the preceding exemplary embodiments, wherein the morselizing screen is frustoconical.
74. The device according to any one of the preceding exemplary embodiments, wherein the morselizing screen rotates along with the rotatable chamber.
75. The device according to any one of the preceding exemplary embodiments, wherein the morselizing screen is stationary relative to the rotatable chamber.
76. The device according to any one of the preceding exemplary embodiments, wherein the morselizing screen extends concentrically around the axis of rotation of the rotatable chamber.
77. The device according to any one of the preceding exemplary embodiments, wherein the morselizing screen is mesh-like.
78. The device according to any one of the preceding exemplary embodiments, wherein the morselizing screen is made of metal or polymer wire material, or a perforated sheet having openings of sufficient size to allow for the morselization of a tissue.
79. The device according to any one of the preceding exemplary embodiments, wherein the morselizing screen is in contact with the first section.
80. The device according to any one of the preceding exemplary embodiments, wherein the morselizing screen is in contact with the second section.
81. The device according to any one of the preceding exemplary embodiments, wherein the morselizing screen is in contact with at least one sidewall.
82. The device according to any one of the preceding exemplary embodiments, further comprising a roller arranged to urge tissue through the morselizing screen.
83. The device according to any one of the preceding exemplary embodiments, wherein the roller comprises a roller axle and a cylinder rotatable about the roller axle.
84. The device according to any one of the preceding exemplary embodiments, wherein the roller axle is formed of stiff wire that extends through the center bore of the cylinder and the wire is secured in a stationary position within the rotatable chamber.
85. The device according to any one of the preceding exemplary embodiments, wherein the roller axle extends from the sidewall of the first section or the second section.
86. The device according to any one of the preceding exemplary embodiments, wherein the roller is present proximate or in contact with the morselizing screen.
87. The device according to any one of the preceding exemplary embodiments, wherein the morselizing screen is rotatable about the axis of rotation of the rotatable chamber while the roller axle remains stationary.
88. The device according to any one of the preceding exemplary embodiments, wherein the roller is present near the inner surface of the morselizing screen but does not contact the morselizing screen.
89. The device according to any one of the preceding exemplary embodiments, wherein the roller rolls against the inner surface of the morselizing screen.
90. The device according to any one of the preceding exemplary embodiments, further comprising a second morselizing screen, the second morselizing screen having an average aperture size that is less than the morselizing screen, the second morselizing screen present at a greater distance from the axis than the morselizing screen.
91. The device according to any one of the preceding exemplary embodiments, further comprising a second morselizing screen and a second roller, the second roller arranged to urge tissue through the second morselizing screen, the second morselizing screen having an average aperture size that is less than the morselizing screen, the second morselizing screen present at a greater distance from the axis than the morselizing screen.
92. The device according to any one of the preceding exemplary embodiments, further comprising a third morselizing screen, the third morselizing screen having an average aperture size that is less than the second morselizing screen, the third morselizing screen present at a greater distance from the axis than the second morselizing screen.
93. The device according to any one of the preceding exemplary embodiments, further comprising a third morselizing screen and a third roller, the third roller arranged to urge tissue through the third morselizing screen, the third morselizing screen having an average aperture size that is less than the second morselizing screen, the third morselizing screen present at a greater distance from the axis than the second morselizing screen.
94. The device according to any one of the preceding exemplary embodiments, wherein one or more morselizing screens are attached to the second section and one or more roller axles are attached to the first section.
95. The device according to any one of the preceding exemplary embodiments, wherein the device comprises more than one roller and associated morselizing screen, wherein each roller is evenly spaced circumferentially around the axis and at different radial distances from the axis.
96. The device according to any one of the preceding exemplary embodiments, further comprising a retaining screen.
97. The device according to any one of the preceding exemplary embodiments, further comprising a retaining screen configured to restrict the passage of a constituent of biologic tissue therethrough.
98. The device according to any one of the preceding exemplary embodiments, further comprising a retaining screen, wherein the retaining screen is present at a greater radial distance from the axis than any morselizing screen.
99. The device according to any one of the preceding exemplary embodiments, wherein the drive unit couples to the rotatable chamber.
100. The device according to any one of the preceding exemplary embodiments, wherein the drive unit comprises an electric motor configured to rotate the rotatable chamber.
101. The device according to any one of the preceding exemplary embodiments, wherein the drive unit is separable from the rotatable chamber such that the drive unit may be reused without substantial cleaning or sterilization.
102. The device according to any one of the preceding exemplary embodiments, wherein the drive unit is configured to rotate the rotatable chamber such that the g-force acting on a sample within the rotatable chamber is 1000 g or less, 8000 g or less, or 5000 g or less.
103. The device according to any one of the preceding exemplary embodiments, wherein the drive unit is configured to oscillate the rotatable chamber.
104. The device according to any one of the previous exemplary embodiments, wherein the device is configured to allow for addition of a cleaning solution while the rotatable chamber is rotating.
105. A process for processing a sample of biologic tissue comprising the steps of:
   a. placing a sample of biologic tissue at the center of a rotatable chamber, the rotatable chamber having two ends and comprising an annular cavity present between two ends of the rotatable chamber,
   b. rotating the rotatable chamber, thereby producing a centrifugal field,
   c. morselizing at least a portion of the biologic tissue by passing a quantity of the biologic tissue through a morselizing screen present within the rotatable chamber, thereby forming a morselized sample,
   d. stratifying the morselized sample into at least two constituents based on the specific gravity of the constituents, and
   e. depositing a constituent having the highest specific gravity into the annular cavity.
106. The process according to the previous exemplary embodiment, further comprising the step of recovering the desired portion of the sample from the annular cavity.
107. The process according to the previous exemplary embodiment, further comprising the step of recovering the desired portion of the sample from the annular cavity by flushing the annular cavity with a fluid, thereby forcing the desired portion of the sample from a first port, located at a first location of the annular cavity, to a second port, located at a second location of the annular cavity.
108. The process according to any one of the previous exemplary embodiments, further comprising recovering at least a portion of the densest of the constituents from the annular cavity.
109. The process according to any one of the previous exemplary embodiments, further comprising the step of opening a passage to the annular cavity to allow fluid communication between the interior of the rotatable chamber and the annular cavity.
110. The process according to any one of the previous exemplary embodiments, further comprising the step of closing a passage to the annular cavity thereby retaining the constituent having the highest specific gravity in the annular cavity.
111. The process according to any one of the previous exemplary embodiments, further comprising the step of adding a cleaning solution after removing a first fluid constituent from the sample and prior to recovering the desired constituent of the sample.
112. The process according to any one of the previous exemplary embodiments, further comprising the step of adding a cleaning solution to the rotatable chamber while the rotatable chamber is rotating.
113. The process according to any one of the previous exemplary embodiments, further comprising the step of retaining at least a portion of the biologic tissue on a retaining screen.
114. The process according to any one of the previous exemplary embodiments, wherein the process is performed utilizing the device of any one of the previous exemplary embodiments.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

1. A device for processing biologic tissue, the device comprising:
a. a rotatable chamber arranged to rotate about an axis, the rotatable chamber at least partially defined by a first section and a second section, each of the first section and the second section comprising a base, an end, and a sidewall connecting the base and the end, the sidewall comprising at least one inclined surface such that each section decreases in interior width over at least a portion of the distance from its base to its end, the first section and second section positioned opposite one another such that the first section base and the second section base are positioned adjacent to one another, and
b. a drive unit configured to rotate the rotatable chamber about the axis and produce a centrifugal field via rotation of the rotatable chamber about the axis:
wherein at a first position the device comprises a passage defining the entrance to an annular cavity, the annular cavity being present i) in a sidewall of the first section or second section, or ii) between the first section and the second section, and wherein the device is configured such that upon operation at least a portion of a sample of biologic tissue present in the rotatable chamber is stratified into at least two constituent layers as a function of the differing specific gravities of the constituents of the sample of biologic tissue, and wherein at least a portion of one or more of the at least two constituent layers may enter the annular cavity.

2. A device for processing biologic tissue, the device comprising:
a. a rotatable chamber arranged to rotate about an axis and comprising a sidewall connecting the ends of the chamber, wherein the interior of the rotatable chamber varies in width along at least a portion of the distance from a first end to the second end such that the widest point of the rotatable chamber is located between the first end and the second end, the rotatable chamber comprising an annular cavity proximate its widest point, and
b. a drive unit configured to rotate the rotatable chamber about the axis and produce a centrifugal field via rotation of the rotatable chamber about the axis:
wherein the device is configured such that upon operation at least a portion of a sample of biologic tissue present in the rotatable chamber is stratified into at least two constituent layers as a function of the differing specific gravities of the constituents of the sample of biologic tissue, and wherein at least a portion of one or more of the at least two constituent layers may enter the annular cavity.

3. The device according to claim 2, wherein at a first position the device allows fluid communication from the interior of the rotatable chamber to the annular cavity and at a second position there is no fluid communication from the interior of the rotatable chamber to the annular cavity, and

4. The device according to claim 1, wherein at a second position, the passage is closed such that any portion of the sample cannot enter the annular cavity.

5. The device according to claim 3, wherein the device is operable to move from the first position to the second position by translating a section or the rotatable chamber axially along the axis.

6. The device according to claim 1, wherein the device is operable to move from the first position to the second position by closing a first valve that controls fluid communication through the passage to the annular cavity.

7. The device according to claim 1, further comprising a port for accessing the annular cavity from the exterior of the rotatable chamber.

8. The device according to claim 1, wherein the annular cavity comprises the shape of a circular or ellipsoidal ring.

9. The device according to claim 1, wherein the annular cavity comprises the shape of an eccentric ring.

10. The device according to claim 1, wherein the annular cavity has a maximum distance from the axis at a first radial location that is greater than the maximum distance from the axis at a second radial location.

11. The device according to claim 1, wherein the annular cavity comprises an obstruction.

12. The device according to claim 1, wherein the rotatable chamber is at least partially defined by a first section and a second section, and the first section and the second section comprise the shape of a frustum of a cone.

13. The device according to claim 1, wherein the rotatable chamber is at least partially defined by a first section and a second section, and a wall of the first section and a wall of the second section define the passage therebetween.

14. The device according to claim 13, wherein the rotatable chamber is at least partially defined by a first section and a second section, and the device comprises a collar connected to the first section or the second section, the collar comprising the annular cavity.

15. The device according to claim 13, wherein the annular cavity is in fluid communication with a collection container via a port present in the collar.

16. The device according to claim 1, further comprising a morselizing screen present within the rotatable chamber.

17. The device according to claim 1, wherein the morselizing screen is in contact with a sidewall of the rotatable chamber.

18. A process for processing a sample of biologic tissue comprising the steps of:
a placing a sample of biologic tissue at the center of a rotatable chamber, the rotatable chamber having two ends and comprising an annular cavity present between two ends of the rotatable chamber,
b. rotating the rotatable chamber, thereby producing a centrifugal field,
c. morselizing at least a portion of the biologic tissue by passing a quantity of the biologic tissue through a morselizing screen present within the rotatable chamber, thereby forming a morselized sample,
d. stratifying the morselized sample into at least two constituents based on the specific gravity of the constituents, and
e. depositing a constituent having the highest specific gravity into the annular cavity.

19. The process according to claim 18, further comprising recovering at least a portion of the densest of the constituents from the annular cavity.

20. The process according to claim 18, further comprising the step of closing a passage to the annular cavity thereby retaining the constituent having the highest specific gravity in the annular cavity.
